(19) United States
(12) Patent Application Publication
(10) Pub. No.: US 2022/0180973 A1
(43) Pub. Date: Jun. 9, 2022

(54) EARLY DETECTION AND PREDICTION METHOD OF PAN-CANCER

(71) Applicant: Quark Biosciences Taiwan, Inc., Hsinchu County (TW)

(72) Inventors: Shih-Ting Kang, Hsinchu County (TW); Wei-Ming Chen, Hsinchu County (TW)

(73) Assignee: Quark Biosciences Taiwan, Inc., Hsinchu County (TW)

(21) Appl. No.: 17/544,947

(22) Filed: Dec. 8, 2021

Related U.S. Application Data
(60) Provisional application No. 63/122,481, filed on Dec. 8, 2020.

(51) Int. Cl. G16B 40/20; C12N 15/10; C12Q 1/6886

(52) U.S. Cl. CPC G16B 40/20; C12N 15/1096; C12Q 2600/118; C12Q 2600/178; C12Q 2600/156; C12Q 1/6886

(57) ABSTRACT

An early detection and prediction method of pan-cancer is provided, including the following steps. A microRNA expression profile database of cancer patient populations and healthy populations is established, and an early detection and prediction method of pan-cancer is developed based on the microRNA expression profile database. Next, a microRNA expression profile of a liquid biopsy sample of a subject is analyzed via the early detection and prediction method of pan-cancer, so as to determine whether the subject may already have cancer.

### CROSS-REFERENCE TO RELATED APPLICATION

This application claims the priority benefit of U.S. provisional application Ser. No. 63/122,481, filed on Dec. 8, 2020. The entirety of the above-mentioned patent application is hereby incorporated by reference herein and made a part of this specification.

### BACKGROUND OF THE INVENTION

### Field of the Invention

The invention relates to an early detection method, and particularly relates to an early detection and prediction method of pan-cancer.

### Description of Related Art

MicroRNA is a non-coding RNA with a length of about 18 to 25 nucleotides. MicroRNA is highly preserved during the evolution process, and plays a very important role in the regulation of cells. In 1993, miRNA was first discovered in C. elegans. One after another, more and more miRNAs have been discovered in humans and other species. At present, there are about 2,500 known miRNAs in human cells. These miRNAs have been proven to regulate more than 50% messenger RNAs' (mRNAs′) expression. Moreover, abnormal miRNA expression has been proved to be closely related to the formation of many diseases, such as cancer diseases, chronic diseases, and autoimmune diseases.

In the past few years, miRNA has been widely praised and is regarded as a new target for molecular detection. At present, miRNA has also been confirmed to be secreted from cells into the blood and form protein-RNA complexes, ensuring that it will not be degraded by ribonuclease (RNase). Such features have also become very valuable, making free miRNAs in the blood relatively easy to obtain, and may be used as a basis for initial diagnosis of diseases by detecting cell-free miRNA expression profiling. For example, different types of cancers have been confirmed to have their own unique expression profiles of free miRNA, or miRNA signatures, which may be used as the basis for the initial diagnosis of cancer.

For a long time, non-invasive disease detection methods that are convenient and have high diagnostic rate have been a constantly pursued goal of the medical community. Taking cancer as an example, in order to find potentially undetected and early-stage asymptomatic cancers early, cancer screening may be performed to achieve this goal. Cancer screening refers to the process of using examinations, tests, or other methods to identify the possible presence or absence of cancer.

Currently, patients may be tested for cancer via many symptoms or test results. However, the most certain way to diagnose malignant tumors is to confirm the presence of cancer cells by pathologists in biopsy or surgically obtained tissues, which is an invasive detection method.

In addition, tumor marker detection refers to the determination of cancer by detecting changes in special proteins associated with malignant tumor cells. However, the sensitivity and specificity of tumor marker detection is not good, and tumor is often detected when the tumor has already developed to a considerable size or has metastasized to other organs.

Based on the above, the development of a non-invasive, early detection method of pan-cancer, determining whether the subject has cancer, and early diagnosis and treatment are important topics for current research.

### SUMMARY OF THE INVENTION

The invention provides an early detection and prediction method of pan-cancer detecting early-stage cancer by analyzing the miRNA expression profile of a liquid biopsy sample of a subject.

An early detection and prediction method of pan-cancer of the invention includes the following steps. A miRNA expression profile database of cancer patient populations and healthy populations is established, and an early detection and prediction method of pan-cancer is developed based on the miRNA expression profile database. The early detection and prediction method of pan-cancer is a prediction method constructed by the basis of SVM. The prediction method is established by using the miRNA expression profiles of liquid biopsy samples of cancer patient populations and healthy populations through the following steps: a data normalization, an imputation, a data scaling, a predictive modeling, and a cross-validation. After the prediction method is established, the miRNA expression profile of the liquid biopsy sample of the subject is used as a basis to predict the subject's initial diagnosis of cancer. A confusion matrix of prediction results thereof may be used to evaluate the effectiveness of the early detection and prediction method of pan-cancer.

In an embodiment of the invention, the miRNA expression profile is determined by qPCR, sequencing, microarray, or RNA-DNA hybrid capture technology.

In an embodiment of the invention, the miRNA expression profile is determined by performing qPCR on a cDNA synthesized from a miRNA in the liquid biopsy sample.

In an embodiment of the invention, the miRNA expression profile includes an expression level of a plurality of miRNAs.

In an embodiment of the invention, the plurality of miRNAs include at least 167 miRNAs.

In an embodiment of the invention, a type of the early detection of cancer includes head and neck cancer, lung cancer, or breast cancer.

In an embodiment of the invention, the liquid biopsy sample includes plasma, serum, or urine.

In an embodiment of the invention, the normalization is used to make an experimental data distribution of each sample consistent.

In an embodiment of the invention, the imputation is used to correct a biomarker with no signal to a maximum value of a cycle threshold (Cq) of a microRNA biomarker in all samples.

In an embodiment of the invention, the data scaling is used to normalize a numerical range of a data so that the data has zero-mean and unit-variance.

Based on the above, the invention provides a non-invasive, early detection method of pan-cancer, in which the miRNA expression profile of a liquid biopsy sample of a subject is analyzed via the early detection and prediction method of pan-cancer. Therefore, early cancer screening and diagnosis may be performed in time and efficiently, and the convenience and detection rate of conventional cancer screening methods may be improved.

In order to make the aforementioned features and advantages of the invention more comprehensible, embodiments are described in detail below.

### BRIEF DESCRIPTION OF THE DRAWINGS

None

### DESCRIPTION OF THE EMBODIMENTS

In the following, the embodiments of the invention are described in detail. However, the embodiments are exemplary, and the invention is not limited thereto.

The invention provides an early detection and prediction method of pan-cancer. In the following, the terms used in the specification are defined first.

‘cDNA’ (complementary DNA) refers to complementary DNA generated by performing reverse transcription on an RNA template using reverse transcriptase.

‘qPCR’ or ‘real-time quantitative PCR’ (real-time quantitative polymerase chain reaction) refers to an experimental method of using PCR to amplify and quantify target DNA at the same time. Quantification is performed using a plurality of measuring chemical substances (including, for example, fluorescent dye of SYBR® green or fluorescent report oligonucleotide probe of Taqman probe), and real-time quantification is performed with the amplified DNA accumulated in the reaction after every amplification cycle.

The term ‘expression’ refers to the transcription and/or accumulation of RNA molecules in a biological sample, such as a liquid biopsy sample. In this context, the term ‘miRNA expression’ refers to one or a plurality of miRNAs in a biological sample, and the miRNA expression may be detected by using a suitable method known in the art.

The term ‘microribonucleic acid’ (microRNA′ or ‘miRNA’) refers to a type of non-coding RNA with a length of about 18 to 25 nucleotides derived from an endogenous gene. miRNA acts as a post-transcriptional regulator of gene expression via base pairing with the 3′-untranslated region (UTR) of the target mRNA thereof for mRNA degradation or translation inhibition.

The terms ‘nucleic acid’, ‘nucleotide’, and ‘polynucleotide’ are used interchangeably and refer to a polymer of DNA or RNA in single-stranded or double-stranded form. Unless stated otherwise, these terms encompass polynucleotides containing known analogs of natural nucleotides that have binding properties similar to a reference nucleic acid and are metabolized in a manner similar to naturally occurring nucleotides.

The term ‘primer’ refers to an oligonucleotide used to initiate the synthesis of complementary nucleic acid strands when under conditions that induce the synthesis of a primer extension product, for example, when the oligonucleotide is placed in the presence of a nucleotide and a polymerization inducer (such as DNA or ribonucleic acid polymerase) and at a suitable temperature, pH, metal ion concentration, and salt concentration.

The term ‘probe’ refers to a structure including a polynucleotide and contains a nucleic acid sequence complementary to a nucleic acid sequence present in a target nucleic acid analyte (for example, a nucleic acid amplification product). The polynucleotide region of the probe may be composed of DNA and/or RNA and/or synthetic nucleotide analogs. The length of the probe is usually compatible with all or part of the target sequence used for the specific detection of the target nucleic acid.

The term ‘targeting’ refers to the selection of a suitable nucleotide sequence hybridizing with a nucleic acid sequence of interest.

An early detection and prediction method of pan-cancer is provided, including the following steps. A miRNA expression profile database of cancer patient populations and healthy populations is established, and an early detection and prediction method of pan-cancer is developed based on the miRNA expression profile database. Next, a miRNA expression profile of a liquid biopsy sample of a subject is analyzed by the early detection and prediction method of pan-cancer to be used as a basis for the initial diagnosis of cancer. In more detail, the liquid biopsy sample may include plasma, serum, or urine, but the invention is not limited thereto.

The early detection and prediction method of pan-cancer of the invention uses a supervised learning support vector machine (referred to as SVM) as a modeling basis. The original SVM was invented in 1963 by Vladimir N. Vapnik and Alexey Ya. Chervonenkis. In 1992, Bernhard E. Boser, Isabelle M. Guyon, and Vladimir N. Vapnik proposed a method of maximum-margin hyperplanes by kernel trick to build a nonlinear classifier. The predecessor of the current SVM classifier standard was proposed by Corinna Cortes and Vladimir N. Vapnik in 1993 and published in 1995. (https://link.springer.com/article/10.1007%2FBF00994018)

In the present embodiment, a disease-related miRNA information database was established based on more than 30,000 articles, and 167 miRNAs highly related to cancer were screened out. The 167 miRNAs screened out that are highly related to cancer are shown in Table 1 below.

TABLE 1
167 miRNA biomarkers

| | | | | |
|---|---|---|---|---|
| hsa-let-7d-3p | hsa-miR-214-3p | hsa-let-7b-5p | hsa-miR-20b-5p | hsa-miR-451a |
| hsa-miR-101-3p | hsa-miR-215-5p | hsa-let-7c-5p | hsa-miR-210-3p | hsa-miR-23a-3p |
| hsa-miR-122-5p | hsa-miR-21-5p | hsa-let-7d-5p | hsa-miR-217 | hsa-let-7a-5p |
| hsa-miR-1254 | hsa-miR-216a-5p | hsa-let-7f-5p | hsa-miR-221-3p | hsa-miR-103a-3p |
| hsa-miR-125b-5p | hsa-miR-222-3p | hsa-let-7g-5p | hsa-miR-223-3p | hsa-miR-16-5p |
| hsa-miR-126-3p | hsa-miR-22-3p | hsa-miR-100-5p | hsa-miR-27a-3p | hsa-miR-191-5p |
| hsa-miR-1290 | hsa-miR-224-5p | hsa-miR-106a-5p | hsa-miR-29a-3p | hsa-miR-423-3p |
| hsa-miR-129-5p | hsa-miR-24-3p | hsa-miR-106b-5p | hsa-miR-29b-3p | hsa-miR-423-5p |
| hsa-miR-140-5p | hsa-miR-26a-5p | hsa-miR-10a-5p | hsa-miR-29c-3p | hsa-miR-93-5p |
| hsa-miR-142-3p | hsa-miR-28-3p | hsa-miR-10b-5p | hsa-miR-302d-3p | hsa-miR-425-5p |
| hsa-miR-143-3p | hsa-miR-299-5p | hsa-miR-1248 | hsa-miR-30a-5p | hsa-miR-1228-5p |
| hsa-miR-144-3p | hsa-miR-29a-5p | hsa-miR-125a-5p | hsa-miR-30c-5p | |
| hsa-miR-145-5p | hsa-miR-30b-5p | hsa-miR-127-3p | hsa-miR-30d-5p | |
| hsa-miR-146a-5p | hsa-miR-31-5p | hsa-miR-128-3p | hsa-miR-30e-5p | |
| hsa-miR-150-5p | hsa-miR-326 | hsa-miR-130a-3p | hsa-miR-31-3p | |
| hsa-miR-151a-3p | hsa-miR-335-5p | hsa-miR-130b-3p | hsa-miR-320a | |
| hsa-miR-152-3p | hsa-miR-338-5p | hsa-miR-133a-3p | hsa-miR-330-5p | |
| hsa-miR-155-5p | hsa-miR-34a-5p | hsa-miR-133b | hsa-miR-376c-3p | |
| hsa-miR-15a-5p | hsa-miR-361-5p | hsa-miR-134-5p | hsa-miR-411-5p | |
| hsa-miR-15b-5p | hsa-miR-372-3p | hsa-miR-135a-5p | hsa-miR-429 | |
| hsa-miR-17-3p | hsa-miR-373-3p | hsa-miR-135b-5p | hsa-miR-4306 | |
| hsa-miR-181a-5p | hsa-miR-375 | hsa-miR-1-3p | hsa-miR-450a-5p | |
| hsa-miR-181b-5p | hsa-miR-378a-5p | hsa-miR-141-3p | hsa-miR-486-5p | |
| hsa-miR-182-5p | hsa-miR-382-5p | hsa-miR-146b-5p | hsa-miR-518a-5p | |
| hsa-miR-183-5p | hsa-miR-409-3p | hsa-miR-17-5p | hsa-miR-520b | |
| hsa-miR-193a-3p | hsa-miR-425-3p | hsa-miR-18a-5p | hsa-miR-539-5p | |
| hsa-miR-1972 | hsa-miR-452-3p | hsa-miR-18b-5p | hsa-miR-625-5p | |
| hsa-miR-197-3p | hsa-miR-483-5p | hsa-miR-192-5p | hsa-miR-652-3p | |
| hsa-miR-198 | hsa-miR-484 | hsa-miR-193b-3p | hsa-miR-660-5p | |
| hsa-miR-199a-5p | hsa-miR-499a-5p | hsa-miR-195-5p | hsa-miR-663a | |
| hsa-miR-19a-3p | hsa-miR-500a-5p | hsa-miR-196a-5p | hsa-miR-718 | |
| hsa-miR-19b-3p | hsa-miR-574-3p | hsa-miR-196b-5p | hsa-miR-7-5p | |
| hsa-miR-202-3p | hsa-miR-574-5p | hsa-miR-1973 | hsa-miR-760 | |
| hsa-miR-203a-3p | hsa-miR-579-3p | hsa-miR-199a-3p | hsa-miR-885-5p | |
| hsa-miR-205-5p | hsa-miR-589-5p | hsa-miR-200a-3p | hsa-miR-92a-3p | |
| hsa-miR-206 | hsa-miR-593-5p | hsa-miR-200b-3p | hsa-miR-95-3p | |
| hsa-miR-20a-5p | hsa-miR-596 | hsa-miR-200c-3p | hsa-miR-9-5p | |
| hsa-miR-2114-3p | hsa-miR-601 | hsa-miR-208b-3p | hsa-miR-99b-5p | |
| hsa-miR-940 | hsa-miR-1225-3p | hsa-miR-223-5p | hsa-miR-4772-3p | |

In the present embodiment, the method of detecting miRNA in plasma includes the following steps:

1. Collection of Blood Sample

A blood sampler's skin was wiped with alcohol on the blood collection site, and a tourniquet was tied 5 cm to 15 cm above the blood collection site with a slip knot. 10 ml of whole blood was drawn into a K₂EDTA BD Vacutainer tube using a 19 G to 22 G needle. When blood flows into the blood collection tube, the tourniquet should be released immediately. After the blood draw was completed, the blood collection tube was immediately turned upside down and mixed lightly 5 to 8 times to ensure that the anticoagulant was fully functional. The blood collection tube was stored at room temperature, and the plasma separation step was completed within one hour after blood collection.

2. Plasma Separation Method

The blood collection tube was placed on a swinging-bucket rotor and centrifuged at 1200×g for 10 minutes at room temperature. After centrifugation was complete, the supernatant was taken out to a new 15 ml centrifuge tube. The 15 ml centrifuge tube was pipetted 5 times to ensure even mixing, and then was evenly divided into 1.5 ml DNase/RNase-free Eppendorf, and centrifuged at 12,000×g for 10 minutes at room temperature. After centrifugation was complete, the supernatant was taken out and transferred to a new 15 ml centrifuge tube to avoid taking the white sediment at the bottom of the 1.5 ml Eppendorf. The supernatant was pipetted 5 times to ensure even mixing, dispensed into 1.5 ml DNA LoBind Tubes (Eppendorf, 22431021), and stored immediately in a refrigerator at −80° C.

3. miRNA Extraction Method

The plasma sample was taken out from the refrigerator at −80° C., thawed on ice, and subjected to an experiment in accordance with the operation manual provided by Qiagen miRNeasy Serum/Plasma Kit after thawing, and then reconstituted with 30 μl nuclease-free water.

4. cDNA Synthesis

A suitable amount of miRNA was taken and a reverse transcription reaction was performed using Quarkbio microRNA Universal RT kit to synthesize cDNA.

5. qPCR Experiment

A suitable amount of cDNA was taken to perform qPCR experiment with the operation manual provided by Quarkbio mirSCAN Panelchip®.

In the present embodiment, the healthy subjects and cancer patients in the selected samples were classified by a physician. Each type of cancer patient was a non-metastatic patient in stage 1 to 2, had just been diagnosed, and had not received treatment. Patients who were determined as newly diagnosed cancer patients had their blood drawn before treatment, and the expression level of 167 miRNAs in the plasma was detected by the above method. Those who were determined to be healthy also had their blood drawn to detect the expression level of 167 miRNAs in the plasma by the above method.

In the present embodiment, the identification of early cancer screening may include cancer or healthy subjects, for example, may include head and neck cancer, lung cancer, breast cancer, or healthy subjects. However, the invention is not limited thereto, and may also include other cancers, tumor risks, or risk factors for cancer. More specifically, for the miRNA database for different cancer populations and healthy populations and the early detection and prediction method of pan-cancer established thereby, according to the miRNA expression profile of the subject, the diagnosis prediction may be divided into breast cancer, head and neck cancer, lung cancer, or healthy subjects, but the invention is not limited thereto. More specifically, the expression profile of miRNA may be determined by, for example, qPCR, sequencing, microarray, or RNA-DNA hybrid capture technology, preferably, for example, by performing qPCR on cDNA synthesized from miRNA in a liquid biopsy sample.

In the present embodiment, the early detection and prediction method of pan-cancer is an algorithm constructed based on SVM. In more detail, the following steps are performed to construct the prediction method: data normalization, imputation, data scaling, predictive modeling, and cross-validation.

### Data Normalization

In order to make the distribution the same in statistical properties, the data may be normalized by quantile normalization, as described by Bolstad et al. in ‘A comparison of normalization methods for high density oligonucleotide array data based on variance and bias’, (Bioinformatics, 2003, 19(2):185-193).

After experimental testing, all experimental raw data of each sample was normalized, so that the experimental data distribution of each sample was consistent.

### Imputation

If the internal control group of the experimental design was not affected by any unexpected factors, any miRNA biomarker without signal in the experimental detection was treated as no signal and imputated. The method of imputation corrected a biomarker with no signal to the maximum value of the cycle threshold (Cq) of the microRNA biomarker in all samples.

### Data Scaling

To ensure that the target function worked properly, the numerical range of the data may be normalized so that the data had zero-mean and unit-variance.

### Predictive Modeling

After the data was scaled, the data may be used to further construct a supervised learning classification model having Support Vector Machine (SVM). 121 samples (38 healthy subjects, 18 head and neck cancer, 53 lung cancer, 12 breast cancer) were used to train the model, and k-fold cross-validation was used to evaluate the feasibility of the model and find the best parameters of the model.

### Cross-Validation

The k-fold cross-validation method (for example, 10-fold cross-validation) may be used to evaluate the cancer detection performance of the early detection and prediction method of cancer before the final completion. In k-fold cross-validation, the original samples were randomly divided into k equally divided sub-samples. Among the k sub-samples, one of the sub-samples was reserved as the validation dataset for testing the model, and the remaining k−1 sub-samples were used as training dataset. Subsequently, the cross-validation process was repeated k times (folds), wherein each of the k sub-samples was used exactly once as the validation dataset. The k results from equally divided sub-samples may then be averaged (or otherwise combined) to produce a single estimate value. After validation and optimization, an early detection and prediction method for cancer was established. According to the following confusion matrix, it may be known that the cross-validation prediction result of the model, sensitivity=80.62%, specificity=93.32%, positive predictive value=85.47%, negative predictive value=93.57%, and accuracy=82.64%. The confusion matrix had two dimensions (actual and predicted) contingency tables, and both dimensions had the same set of patient types. The type of actual classification of patients was equal to the predicted type of the model, i.e., true positive and true negative. On the contrary, the actual type was not equal to the predicted type, i.e., false positive and false negative.

| | | Patient classification | | | | | | | | |
|---|---|---|---|---|---|---|---|---|---|---|
| | | | Head and | | | | | | | |
| | | Healthy | neck | Lung | Breast | | | | | |
| | | populations | cancer | cancer | cancer | SEN | SPE | PPV | NPV | ACC |
| Model | Healthy | 34 | 3 | 7 | 0 | 89.47% | 87.95% | 77.27% | 94.81% | — |
| prediction | populations | | | | | | | | | |
| result | Head and | 0 | 12 | 2 | 1 | 66.67% | 97.09% | 80.00% | 94.34% | — |
| | neck cancer | | | | | | | | | |
| | Lung cancer | 4 | 3 | 44 | 1 | 83.02% | 88.24% | 84.62% | 86.96% | — |
| | Breast cancer | 0 | 0 | 0 | 10 | 83.33% | 100.00% | 100.00% | 98.20% | — |
| | | | | | | 80.62% | 93.32% | 85.47% | 93.57% | 82.64% |

Sensitivity (SEN) = TP/(TP + FN)
Specificity (SPE) = TN/(TN + FP)
Positive Predictive Value (PPV) = TP/(TP + FP)
Negative Predictive Value (NPV) = TN/(TN + FN)
Accuracy (ACC) = (TP + TN)/(TP + FP + TN + FN)
TP = True Positive
FP = False Positive
FN = False Negative
TN = True Negative

The following content is used to prove that the early detection and prediction method of pan-cancer proposed by the invention may detect cancer in time and efficiently. It must be noted that the following content is the same as the miRNA detecting experimental method of the above embodiment.

The miRNA expression profile of the plasma sample of the subjects was detected for prediction via the early detection and prediction method of pan-cancer. The confusion matrix of prediction results was used to evaluate the effectiveness of the early detection and prediction method.

A total of 64 subjects were selected as test dataset (37 healthy subjects, 27 lung cancer). According to the following confusion matrix, it may be learned that the prediction result of the model, sensitivity=83.98%, specificity=92.28%, positive predictive value=57.03%, negative predictive value=92.08%, and accuracy=84.38%.

| | | Patient classification | | | | | | | | |
|---|---|---|---|---|---|---|---|---|---|---|
| | | | Head and | | | | | | | |
| | | Healthy | neck | Lung | Breast | | | | | |
| | | populations | cancer | cancer | cancer | SEN | SPE | PPV | NPV | ACC |
| Model | Healthy | 32 | 0 | 5 | 0 | 86.49% | 81.48% | 86.49% | 81.48% | — |
| prediction | populations | | | | | | | | | |
| result | Head and | 1 | 0 | 0 | 0 | — | 98.44% | 0.00% | 100.00% | — |
| | neck cancer | | | | | | | | | |
| | Lung cancer | 4 | 0 | 22 | 0 | 81.48% | 89.19% | 84.62% | 86.84% | — |
| | Breast cancer | 0 | 0 | 0 | 0 | — | 100.00% | — | 100.00% | — |
| | | | | | | 83.98% | 92.28% | 57.03% | 92.08% | 84.38% |

Sensitivity (SEN) = TP/(TP + FN)
Specificity (SPE) = TN/(TN + FP)
Positive Predictive Value (PPV) = TP/(TP + FP)
Negative Predictive Value (NPV) = TN/(TN + FN)
Accuracy (ACC) = (TP + TN)/(TP + FP + TN + FN)
TP = True Positive
FP = False Positive
FN = False Negative
TN = True Negative

Based on the above, the invention provides a non-invasive early detection and prediction method of pan-cancer, in which the miRNA expression profile of a liquid biopsy sample of a subject is analyzed via the early detection and prediction method of pan-cancer. Therefore, early cancer screening may be performed in time and efficiently, and the convenience and detection rate of the conventional early cancer detecting technology may be improved, and personalized professional cancer detection and monitoring may be provided.

Although the invention has been described with reference to the above embodiments, it will be apparent to one of ordinary skill in the art that modifications to the described embodiments may be made without departing from the spirit of the disclosure. Accordingly, the scope of the disclosure is defined by the attached claims not by the above detailed descriptions.

What is claimed is:

1. An early detection and prediction method of pan-cancer, comprising:
establishing a miRNA expression profile database of cancer patient populations and healthy populations; and
establishing, based on SVM, by the following steps: a data normalization, an imputation, a data scaling, a predictive modeling, and a cross-validation,
wherein a miRNA expression profile of a liquid biopsy sample of a subject is analyzed by the early detection and prediction method of pan-cancer to be used as a basis for an initial diagnosis of cancer.

2. The early detection and prediction method of pan-cancer of claim 1, wherein the miRNA expression profile is determined by qPCR, sequencing, microarray, or RNA-DNA hybrid capture technology.

3. The early detection and prediction method of pan-cancer of claim 2, wherein the miRNA expression profile is determined by performing qPCR on a cDNA synthesized from a miRNA in the liquid biopsy sample.

4. The early detection and prediction method of pan-cancer of claim 1, wherein the miRNA expression profile comprises an expression level of a plurality of miRNAs.

5. The early detection and prediction method of pan-cancer of claim 4, wherein the plurality of miRNAs comprise at least 167 miRNAs.

6. The early detection and prediction method of pan-cancer of claim 1, wherein a type of the early detection of cancer comprises head and neck cancer, lung cancer, or breast cancer.

7. The early detection and prediction method of pan-cancer of claim 1, wherein the liquid biopsy sample comprises plasma, serum, or urine.

8. The early detection and prediction method of pan-cancer of claim 1, wherein the normalization is used to make an experimental data distribution of each sample consistent.

9. The early detection and prediction method of pan-cancer of claim 1, wherein the imputation is used to correct a biomarker without a signal to a maximum value of a cycle threshold (Cq) of a miRNA biomarker expression in all samples.

10. The early detection and prediction method of pan-cancer of claim 1, wherein the data scaling is used to normalize a numerical range of a data so that the data has zero-mean and unit-variance.
